(19) United States
(12) Patent Application Publication
(10) Pub. No.: US 2005/0043413 A1
(43) Pub. Date: Feb. 24, 2005

(54) Active ingredient for producing a substance containing isocitrate for influencing the coagulation tendency of blood

(75) Inventors: Wolf Bertling, Erlangen (DE); Mats Ranby, Vreta Kloster (SE)

Correspondence Address: LERNER AND GREENBERG, PA, P O BOX 2480, HOLLYWOOD, FL, 33022-2480, US

(73) Assignee: responsif GmbH

(21) Appl. No.: 10/919,042

(22) Filed: Aug. 16, 2004

Related U.S. Application Data
(63) Continuation of application No. PCT/EP03/01437, filed on Feb. 13, 2003.

(30) Foreign Application Priority Data
Feb. 15, 2002 (DE) 102 06 648.5

(51) Int. Cl. A61K 31/19

(52) U.S. Cl. 514/574

(57) ABSTRACT

An active ingredient for producing a medicament for inhibiting the coagulation of blood, and a corresponding medicament are provided. The active ingredient is isocitrate or an active derivative thereof.

### CROSS-REFERENCE TO RELATED APPLICATIONS

This is a continuing application, under 35 U.S.C. § 120, of copending international application No. PCT/EP03/01437, filed Feb. 13, 2003, which designated the United States; this application also claims the priority, under 35 U.S.C. § 119, of German patent application No. 102 06 648.5, filed Feb. 15, 2002; the prior applications are herewith incorporated by reference in their entirety.

### BACKGROUND OF THE INVENTION

1. Field of the Invention

The invention relates to an active ingredient for producing a medicament for influencing the coagulability of blood. The threat of blood loss resulting from injury to vessels of the vascular system is normally prevented by a physiological reaction, which is referred to as hemostasis. This entails an interaction of the platelet system, plasma clotting factors and the injured vessels so that the blood which is liquid in the normal blood circulation forms a barrier to prevent escape of blood.

Clotting of blood is based on a multistage process. In a first stage, the proenzyme prothrombin is converted into thrombin, a precondition for which is activated factor X as conversion factor. Factor X is activated by an extrinsic or an intrinsic reaction pathway. In the extrinsic reaction pathway the phospholipoprotein which occurs in all tissues forms a complex with factor VII and calcium ions as soon as it comes into contact with blood plasma. This complex activates factor X, forming factor Xa. In the intrinsic reaction pathway, initially factor XII is activated by the kallikreinkinin system and then, together with Fitzgerald factor, activates factor XI, which in turn converts factor IX into its active form. Factor IXa, factor VIII and calcium ions form together with phospholipid micelles (platelet factor 3) a complex which is able to activate factor X. In both reaction pathways, factor Xa forms with factor V, calcium ions and phospholipid micelles a complex which catalyzes the conversion of prothrombin into thrombin. Thrombin cleaves two fibrinopeptides off fibrinogen molecules. The fibrin monomers which are formed polymerize with crosslinking, resulting in a stable fibrin clot.

However, inhibition of coagulability is desired in patients with artificial heart valves or artificial blood vessels, with thromboses, embolisms or cardiac arrhythmias, for preparing for and performing operations in cardiac surgery, after myocardial infarctions or bypass operations. For this purpose, substances with coagulationinhibiting properties are added to the patient's blood. One group of these substances, which are referred to as anticoagulants, prevents either the formation of enzymes involved in clotting, or inhibits the effects thereof. A second group alters the reaction conditions necessary for coagulation so that the latter is inhibited. These reaction conditions include in particular the pH and the concentration of free calcium ions in the blood. Examples of anticoagulants of the first group are heparin and hirudin, and examples of the second group are citrates and oxalates.

It is known that some anticoagulants of the second group reduce the concentration of free calcium ions in the blood, called the calcium ion activity. Since the presence of calcium ions in a particular concentration is an essential condition both for the extrinsic and for the intrinsic clotting pathway, an inhibition of both clotting pathways and thus of clotting overall is achieved. This result is based on the ability of these anticoagulants to complex calcium ions and, in this way, to reduce the concentration thereof in the blood. However, calcium ions are not only a constituent of blood but are also essentially important for further physiological functions. An adequate calcium ion activity is a precondition for the action of numerous hormones and enzymes. A permanent reduction in the concentration of free calcium ions in the blood may therefore have disadvantageous consequences.

The effect of citrate, an anticoagulant of the second group, is probably not based, or not exclusively based, on its ability to bind calcium ions. It is therefore suitable in principle for inhibiting the clotting of blood without at the same time reducing the concentration of free calcium ions substantially below the natural level thereof. It is moreover possible to maintain the natural calcium ion concentration at its natural level by adding calcium ions.

International PCT publication WO 98/55129 discloses that administration of banked blood provided with an addition of citrate to patients with liver dysfunctions may lead to cramps and gastrointestinal symptoms. It is proposed to use isocitrate for inhibiting the coagulation of blood in vitro, e.g. of banked blood.

### SUMMARY OF THE INVENTION

It is accordingly an object of the invention to provide an active ingredient for producing a substance with isocitratte for influencing the coagulation tendency of blood which overcomes the above-mentioned disadvantages of the heretofore-known devices and methods of this general type and which provides for an active ingredient for producing a coagulation-inhibiting medicament and a medicament which contains the active ingredient.

With the foregoing and other objects in view there is provided, in accordance with the invention, a method of producing a medicament for influencing a coagulability of blood, the method which comprises: providing as an active ingredient isocitrate or an active derivative thereof.

In accordance with an added feature of the invention, the active ingredient is trisodium isocitrate. In a preferred embodiment of the invention, there is added isocitric acid to the medicament. A preferred embodiment of the medicament additionally comprises citrate. Further, the medicament additionally may comprise calcium compounds.

In accordance with another feature of the invention, the active ingredient inhibits the formation of enzymes in the intrinsic and extrinsic clotting pathway.

In accordance with a further feature of the invention, the active ingredient is used to treat thromboses, embolisms, cardiac arrhythmias and myocardial infarctions. In a preferable application, the active ingredient is used to treat patients with artificial heart valves and artificial blood vessels.

With the above and other objects in view there is also provided, in accordance with the invention, a medicament for influencing a coagulability of blood, comprising, as an active ingredient, isocitrate or an active derivative thereof.

The invention provides for the use of an active ingredient for producing a medicament for influencing, in particular for inhibiting, the coagulation or coagulability of blood, where the active ingredient is isocitrate or an active derivative thereof. The isocitrate is preferably trisodium isocitrate.

The active ingredient is suitable in an outstanding manner for inhibiting the coagulation of the blood circulating in the blood circulation, i.e. in vivo. This effect is based not just on the reduction in the concentration of free calcium ions in the blood. It is assumed that the inhibitory effect of isocitrate is surprisingly based on an inhibition of the formation of enzymes involved in the intrinsic or extrinsic clotting pathway, with both clotting pathways being inhibited. On use of isocitrate as active ingredient it is possible to maintain the calcium ion activity in the blood at the natural, i.e. physiological, level. Compensation for the calcium ions bound by isocitrate is possible by using compounds which release calcium ions in the blood. Calcium salts are suitable, for example, for this purpose.

It is possible by using the proposed active ingredient to produce a medicament which inhibits the coagulation of the blood circulating in the blood circulation. The medicament is particularly suitable for the treatment of disorders in which it is necessary to reduce the coagulability of blood. This is the case in particular in the treatment of thromboses, embolisms, cardiac arrhythmias and myocardial infarctions. In addition, the medicament can be used for the treatment of patients with artificial heart valves and artificial blood vessels.

If necessary, the anticoagulant effect of the isocitrate can be enhanced by addition of citrate. The pH of the blood can be reduced by adding isocitric acid or citric acid, by which means it is likewise possible to achieve an increase in the anticoagulant effect of isocitrate. The reduction in pH brought about on use of isocitrate as active ingredient can also be compensated by addition of suitable compounds the pH. Weak bases, for example, are suitable for this purpose. Although the invention is illustrated and described herein as embodied in an active ingredient for producing a substance containing isocitrate for influencing the coagulation tendency of blood, it is nevertheless not intended to be limited to the details shown, since various modifications and structural changes may be made therein without departing from the spirit of the invention and within the scope and range of equivalents of the claims.

The specific implementation of the invention, however, together with additional objects and advantages thereof will be best understood from the following description of specific examples embodying the invention.

### EXAMPLE 1

Trisodium DL-isocitrate was injected directly into the blood circulation of six-week old female Balb/c mice. The citrate concentration in the blood after the injection was about 20 mM. The concentration of free calcium ions, the pH, the prothrombin time (PT) and the activated partial thromboplastin time are determined at regular intervals. The results revealed a clotting time which was longer than in an experimental group without citrate injection.

### EXAMPLE 2

Trisodium DL-isocitrate was injected directly into the blood circulation of six-week old female Balb/c mice. The citrate concentration in the blood after the injection was about 20 mM. The concentration of free calcium ions was measured immediately after the citrate injection and adjusted to 1.5 mM by giving calcium ions, which corresponds approximately to the physiological level. The pH, the prothrombin time (PT) and the activated partial thromboplastin time are determined at regular intervals. The results revealed a clotting time which was longer than in a control group without citrate injection.

1. In a method of producing a medicament for influencing a coagulability of blood, the method which comprises: providing as an active ingredient isocitrate or an active derivative thereof.

2. The method according to claim 1, wherein the active ingredient is trisodium isocitrate.

3. The method according to claim 1, which-comprises adding isocitric acid to the medicament.

4. The method according to claim 1, which comprises adding citrate to the medicament.

5. The method according to claim 1, which comprises adding calcium compounds to the medicament.

6. The method according to claim 1, wherein the active ingredient is configured to inhibit a formation of enzymes in an intrinsic and extrinsic clotting pathway.

7. A treatment method, which comprises providing a medicament containing, as an active ingredient, isocitrate or an active derivative thereof, and treating therewith one of thromboses, embolisms, cardiac arrhythmias, and myocardial infarctions.

8. A treatment method, which comprises providing the medicament according to claim 9 and treating with the active ingredient patients with artificial heart valves and artificial blood vessels.

9. A medicament for influencing the coagulability of blood, comprising, as an active ingredient, isocitrate or an active derivative thereof.

10. The medicament according to claim 9, wherein said active ingredient is trisodium isocitrate.

11. The medicament according to claim 9, which further comprises isocitric acid.

12. The medicament according to claim 9, which further comprises citrate.

13. The medicament according to claim 9, which further comprises calcium compounds.

14. The medicament according to claim 9 configured to inhibit a formation of enzymes in an intrinsic and extrinsic clotting pathway.

15. The medicament according to claim 9 configured for treating thromboses, embolisms, cardiac arrhythmias, and myocardial infarctions.

16. The medicament according to claim 9 configured for treating patients with artificial heart valves and artificial blood vessels.
